(19) United States
(12) Patent Application Publication
(10) Pub. No.: US 2013/0051516 A1
(43) Pub. Date: Feb. 28, 2013

(54) NOISE SUPPRESSION FOR LOW X-RAY DOSE CONE-BEAM IMAGE RECONSTRUCTION

(71) Applicants: Dong Yang, Rochester, NY (US); Nathan J. Packard, Rochester, NY (US)

(72) Inventors: Dong Yang, Rochester, NY (US); Nathan J. Packard, Rochester, NY (US)

(73) Assignee: Carestream Health, Inc.

(21) Appl. No.: 13/222,432

(22) Filed: Aug. 31, 2011

(51) Int. Cl. A61B 6/03; G06K 9/00

(52) U.S. Cl. 378/4; 382/131

(57) ABSTRACT

Embodiments of methods and/or apparatus for 3-D volume image reconstruction of a subject, executed at least in part on a computer for use with a digital radiographic apparatus can obtain image data for 2-D projection images over a range of scan angles. For each of the plurality of projection images, an enhanced projection image can be generated. In embodiments of imaging apparatus, CBCT systems, and methods for operating the same can, through a de-noising application based on a different corresponding object, maintain image reconstruction characteristics (e.g., for a prescribed CBCT examination) while reducing exposure dose, reducing noise or increase a SNR while an exposure setting is unchanged.

### FIELD OF THE INVENTION

This invention relates generally to the field of diagnostic imaging and more particularly relates to Cone-Beam Computed Tomography (CBCT) imaging. More specifically, the invention relates to a method for improved noise characteristics in reconstruction of CBCT image content.

### BACKGROUND OF THE INVENTION

Conventional noise is often present in acquired diagnostic images, such as those obtained from computed tomography (CT) scanning and other x-ray systems, and can be a significant factor in how well real intensity interfaces and fine details are preserved in the image. In addition to influencing diagnostic functions, noise also affects many automated image processing and analysis tasks that are crucial in a number of applications.

Methods for improving signal-to-noise ratio (SNR) and contrast-to-noise ratio (CNR) can be broadly divided into two categories: those based on image acquisition techniques (e.g., improved hardware) and those based on post-acquisition image processing. Improving image acquisition techniques beyond a certain point can introduce other problems and generally requires increasing the overall acquisition time. This risks delivering a higher X-ray dose to the patient and loss of spatial resolution and may require the expense of a scanner upgrade.

Post-acquisition filtering, an off-line image processing approach, is often as effective as improving image acquisition without affecting spatial resolution. If properly designed, post-acquisition filtering requires less time and is usually less expensive than attempts to improve image acquisition. Filtering techniques can be classified into two groupings: (i) enhancement, wherein wanted (structure) information is enhanced, hopefully without affecting unwanted (noise) information, and (ii) suppression, wherein unwanted information (noise) is suppressed, hopefully without affecting wanted information. Suppressive filtering operations may be further divided into two classes: a) space-invariant filtering, and b) space-variant filtering.

Three-dimensional imaging introduces further complexity to the problem of noise suppression. In cone-beam CT scanning, for example, a 3-D image is reconstructed from numerous individual scans, whose image data is aligned and processed in order to generate and present data as a collection of volume pixels or voxels. Using conventional diffusion techniques to reduce image noise can often blur significant features within the 3-D image, making it disadvantageous to perform more than rudimentary image clean-up for reducing noise content.

Thus, it is seen that there is a need for improved noise reduction and/or control methods that reduce image noise without compromising sharpness and detail for significant structures or features in the image.

### SUMMARY OF THE INVENTION

Accordingly, it is an aspect of this application to address in whole or in part, at least the foregoing and other deficiencies in the related art.

It is another aspect of this application to provide in whole or in part, at least the advantages described herein.

It is another aspect of this application to implement low dose CBCT imaging systems and imaging methods.

It is another aspect of this application to provide a radiographic imaging apparatus that can include a machine based learning regression device and/or processes using low noise target data compensation relationships that can compensate 2D projection data for 3D image reconstruction.

It is another aspect of this application to provide radiographic imaging apparatus/methods that can provide de-noising capabilities that can decrease noise in transformed 2D projection data, decrease noise in 3D reconstructed radiographic images and/or maintain image quality characteristics such as SNR or resolution at a reduced x-ray dose of a CBCT imaging system.

In one embodiment, a method for digital radiographic 3D volume image reconstruction of a subject, executed at least in part on a computer, can include obtaining image data for a plurality of 2D projection images over a range of scan angles; passing each of the plurality of 2D projection images through a plurality of de-noising filters; receiving outputs of the plurality of de-noising filters as inputs to a machine-based regression learning unit; using the plurality of inputs at the machine-based regression learning unit responsive to an examination setting to determine reduced-noise projection data for a current 2D projection image; and storing the plurality of 2D reduced-noise projection images in a computer-accessible memory.

In another embodiment, a method for digital radiographic 3D volume image reconstruction of a subject, executed at least in part on a computer, can include obtaining cone-beam computed tomography image data at a prescribed exposure setting for a plurality of 2D projection images over a range of scan angles; generating, for each of the plurality of 2D projection images, a lower noise projection image by: (i) providing an image data transformation for the prescribed exposure setting according to image data from a different corresponding subject based on a set of noise-reducing filters; (ii) applying the image data transformation individually to the plurality of 2D projection images obtained by: (a) concurrently passing each of the plurality of 2D projection images through the set of noise-reducing filters; and (b) applying the image data transformation individually to the plurality of first 2D projection images pixel-by-pixel to use the outputs of the set of noise-reducing filters to generate the corresponding plurality of lower noise projection images; and storing the lower noise projection images in a computer-accessible memory.

In another embodiment, a digital radiography CBCT imaging system for digital radiographic 3D volume image reconstruction of a subject, can include a DR detector to obtain a plurality of CBCT 2D projection images over a range of scan angles at a first exposure setting; a computational unit to generate, for each of the plurality of 2D projection images, a reduced-noise 2D projection image, the set of noise-reducing filters to select (i) an image data transformation for a prescribed exposure setting, a corresponding different subject, and a plurality of imaging filters, and (ii) apply the image data transformation individually to the plurality of 2D projection images obtained at the first exposure setting to generate the plurality of reduced-noise 2D projection images; and a processor to store the reduced-noise plurality of 2D projection images in a computer-readable memory.

### BRIEF DESCRIPTION OF THE DRAWINGS

For a further understanding of the invention, reference will be made to the following detailed description of the invention which is to be read in connection with the accompanying drawing, wherein:

FIG. 1 is a schematic diagram showing components and architecture used for conventional CBCT scanning.

FIG. 2 is a logic flow diagram showing the sequence of processes used for conventional CBCT volume image reconstruction.

FIG. 3 is a diagram that shows an architecture of an exemplary machine based regression learning unit that can be used in embodiments of CBCT imaging systems (e.g., trained and/or operationally) according to the application.

FIG. 4 is a logic flow diagram showing a sequence of processes used for image processing according to an embodiment of the application.

FIG. 5 is a diagram that shows an architecture of an exemplary machine based regression learning unit that can be used in embodiments of radiographic imaging systems (e.g., CBCT) according to the application.

FIG. 6 is a diagram that shows a topological flow chart of exemplary artificial neural networks that can be used in embodiments according to the application.

### DESCRIPTION OF EXEMPLARY EMBODIMENTS

The following is a description of exemplary embodiments according to the application, reference being made to the drawings in which the same reference numerals identify the same elements of structure in each of the several figures, and similar descriptions concerning components and arrangement or interaction of components already described are omitted. Where they are used, the terms “first”, “second”, and so on, do not necessarily denote any ordinal or priority relation, but may simply be used to more clearly distinguish one element from another. CBCT imaging apparatus and imaging algorithms used to obtain 3-D volume images using such systems are well known in the diagnostic imaging art and are, therefore, not described in detail in the present application. Some exemplary algorithms for forming 3-D volume images from the source 2-D images, projection images that are obtained in operation of the CBCT imaging apparatus can be found, for example, in Feldkamp L A, Davis L C and Kress J W, 1984, Practical cone-beam algorithm, J Opt Soc Am, A6, 612-619.

In typical applications, a computer or other type of dedicated logic processor for obtaining, processing, and storing image data is part of the CBCT system, along with one or more displays for viewing image results. A computer-accessible memory is also provided, which may be a non-volatile memory storage device used for longer term storage, such as a device using magnetic, optical, or other data storage media. In addition, the computer-accessible memory can comprise an electronic memory such as a random access memory (RAM) that is used as volatile memory for shorter term data storage, such as memory used as a workspace for operating upon data or used in conjunction with a display device for temporarily storing image content as a display buffer, or memory that is employed to store a computer program having instructions for controlling one or more computers to practice method and/or system embodiments according to the present application.

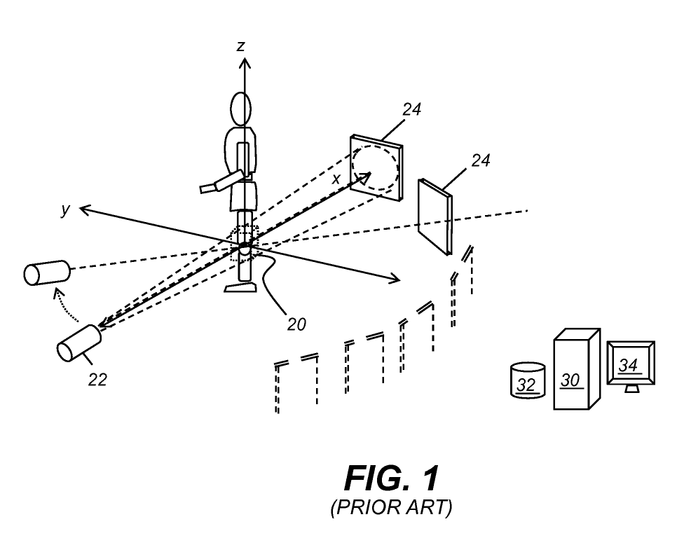

To understand exemplary methods and/or apparatus embodiments according to the present application and problems addressed by embodiments, it is instructive to review principles and terminology used for CBCT image capture and reconstruction. Referring to the perspective view of FIG. 1, there is shown, in schematic form and using exaggerated distances for clarity of description, the activity of an exemplary conventional CBCT imaging apparatus for obtaining the individual 2-D images that are used to form a 3-D volume image. A cone-beam radiation source 22 directs a cone of radiation toward a subject 20, such as a patient or other imaged subject. A sequence of images of subject 20 is obtained in rapid succession at varying angles about the subject over a range of scan angles, such as one image at each 1-degree angle increment in a 200-degree orbit. A DR detector 24 is moved to different imaging positions about subject 20 in concert with corresponding movement of radiation source 22. For example, such corresponding movement can have a prescribed 2D or 3D relationship. FIG. 1 shows a representative sampling of DR detector 24 positions to illustrate how these images are obtained relative to the position of subject 20. Once the needed 2-D projection images are captured in a prescribed sequence, a suitable imaging algorithm, such as FDK filtered back projection or other conventional technique, can be used for generating the 3-D volume image. Image acquisition and program execution are performed by a computer 30 or by a networked group of computers 30 that are in image data communication with DR detectors 24. Image processing and storage is performed using a computer-accessible memory in image data communication with DR detectors 24 such as computer-accessible memory 32. The 3-D volume image or exemplary 2-D image data can be presented on a display 34.

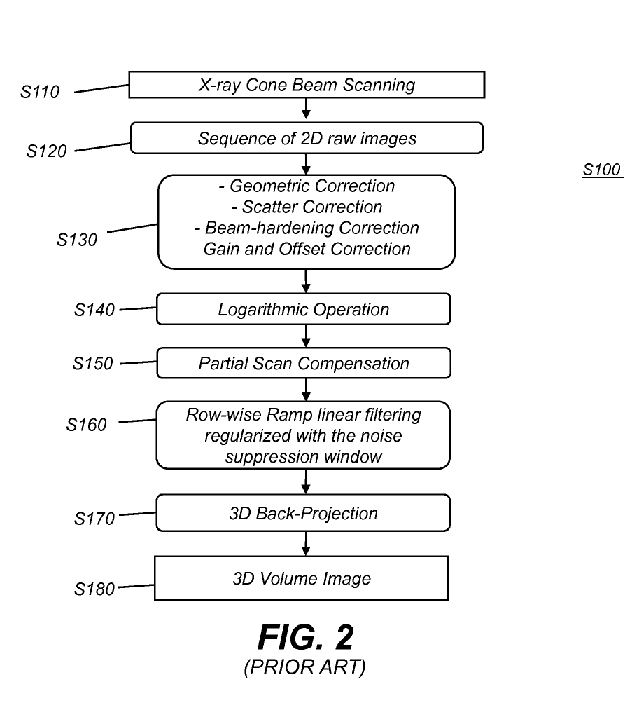

The logic flow diagram of FIG. 2 shows a conventional image processing sequence S100 for CBCT reconstruction using partial scans. A scanning step S110 directs cone beam exposure toward the subject, enabling collection of a sequence of 2-D raw data images for projection over a range of angles in an image data acquisition step S120. An image correction step S130 then performs standard processing of the projection images such as but not limited to geometric correction, scatter correction, gain and offset correction, and beam hardening correction. A logarithmic operation step S140 obtains the line integral data that is used for conventional reconstruction methods, such as the FDK method well-known to those skilled in the volume image reconstruction arts.

An optional partial scan compensation step S150 is then executed when it is necessary to correct for constrained scan data or image truncation and related problems that relate to positioning the detector about the imaged subject throughout the scan orbit. A ramp filtering step S160 follows, providing row-wise linear filtering that is regularized with the noise suppression window in conventional processing. A back projection step S170 is then executed and an image formation step S180 reconstructs the 3-D volume image using one or more of the non-truncation corrected images. FDK processing generally encompasses the procedures of steps S160 and S170. The reconstructed 3-D image can then be stored in a computer-accessible memory and displayed.

Conventional image processing sequence S100 of FIG. 2 has been proven and refined in numerous cases with both phantom and patient images.

It is recognized that in regular x-ray radiographic or CT imaging, the associated x-ray exposure risk to the subjects and operators should reduced or minimized. One way to deliver low dose x-ray to a subject is to reduce the milliampere-second (mAs) value for the radiographic exposure. However, as mAs value decreases, the noise level of the reconstructed image (e.g., CBCT reconstructed image) increases thereby degrading corresponding diagnostic interpretations. X-ray low dose medical imaging will be desirable when clinically acceptable or the same or better image quality (e.g., SNR) can be achieved compared to what current medical x-ray technology can do but with less or significantly less x-ray dose.

Noise is introduced during x-ray generation from the x-ray source and can propagate along as x-rays traverse the subject and then can pass through a subsequent detection system (e.g., radiographic image capture system). Studying noise properties of the transmitted data is a current research topic, for example, in the x-ray Computed Tomography (CT) community. Further, efforts in three categories have been taken to address the low dose x-ray imaging. First, statistical iterative reconstruction algorithms operating on reconstructed image data. Second, roughness penalty based unsupervised nonparametric regressions on the line integral projection data can be used. However, the roughness penalty is calculated based on the adjacent pixels. See, for example, “Sinogram Restoration for Ultra-Low-dose X-ray Multi-slice Helical CT by Nonparametric Regression,” Proc. SPIE Med. Imaging Vol. 6510, pp. 65105L1-10, 2007, by L. Jiang et. al. Third, system dependent parameters can be pulled out to estimate the variance associated with each detector bin by conducting repeated measurement of a phantom under a constant x-ray setting, then adopting penalized weighted least-square (PWLS) method to estimate the ideal line integral projection to achieve the purpose of de-noising. However, estimated variance in the model can be calculated based on the averaging of the neighboring pixel values within a fixed size of square, which may undermine the estimation of the variance, for example, for pixels on the boundary region of two objects. See, for example, “Noise properties of low-dose X-ray CT sonogram data in Radon space,” Proc. SPIE Med. Imaging Vol. 6913, pp. 69131M1-10, 2008, by J. Wang et al.

The first category of iterative reconstruction methods can have an advantage of modeling the physical process of the image formation and incorporating a statistical penalty term during the reconstruction, which can reduce noise while spatial resolution can be fairly maintained. Since the iterative method is computationally intensive, application of the iterative method can be limited by the hardware capabilities. Provided that the sufficient angular samplings as well as approximate noise free projection data are given, the FBP reconstruction algorithm can generate the best images in terms of spatial resolution. An exemplary iterative reconstruction can be found, for example, in “A Unified Approach to Statistical Tomography Using Coordinate Descent Optimization” IEEE Transactions on Image Processing, Vol. 5, No. 3, March 1996.

However, these methodologies use a common property that information from neighboring voxels or pixels whether in reconstruction domain or in projection domain will be used to estimate the noise free centering voxel or pixel. The use of neighboring voxels or pixels is based on the assumption that the neighboring voxels or pixels have some statistical correlations that can be employed (e.g., mathematically) to estimate the mean value of the selected (e.g., centered) pixel.

In contrast to related art methods of noise control, embodiments of DR CBCT imaging systems, computational units and methods according to the application do not use information of neighboring pixels or voxels for reducing or controlling noise for a selected pixel. Exemplary embodiments of DR imaging systems and methods can produce approximate noise-free 2D projection data, which can then be used in reducing noise for or de-noising corresponding raw 2D projection image data. Embodiments of systems and methods according to the application can use CBCT imaging systems using a novel machine learning based unit/procedures for x-ray low dose cone beam CT imaging. In one embodiment, before de-noising, line integral projection data can go through some or all exemplary preprocessing, such as gain, offset calibration, scatter correction, and the like.

In embodiments of imaging apparatus, CBCT imaging systems, and methods for operating the same, de-noising operations can be conducted in the projection domain with comparable or equivalent effect as statistical iterative methods working in the reconstructed image domain when a sufficient angular sampling rate can be achieved. Thus, in exemplary embodiments according to the application, iterations can be in the projection domain, which can reduce or avoid excessive computation loads associated with iteration conducted in reconstructed image domain. Further, variance of line integral projection data at a specific detector pixel can be sufficiently or completely determined by two physical quantities: (1) line integral of the attenuation coefficients along x-ray path; and (2) incident phantom number (e.g., the combination of tube kilovolt peak (kVp) and milliampere seconds (mAs).

Exemplary embodiments described herein take a novel approach to noise reduction procedures by processing the projection data (e.g., 2D) according to a truth image (e.g., first image or representation) prior to reconstruction processing for 3D volume image reconstruction. The truth image can be of a different subject that corresponds to a subject being currently exposed and imaged. In one embodiment, the truth image can be generated using a plurality of corresponding objects.

Repeated measurements generating the projection data at a fixed position and with a constant x-ray exposure parameter can produce an approximate noise-free projection data, which can be almost near truth and can be used for a truth image. Noise or the statistical randomness of noise can be reduced or removed after processing (e.g., averaging or combining) a large number of images (e.g., converted to projection data) of a test object obtained under controlled or identical exposure conditions to generate the approximate noise-free projection data. For example, such approximate noise free data can be acquired by averaging 1000 projection images, in which an object is exposed with the same x-ray parameters for 1000 times. Alternatively, such approximate noise free data can be acquired by averaging more or fewer projection images such as 200, 300, 500, 750 or 5000 projection images.

Unlike related art de-noising methods, embodiments of CBCT imaging systems and methods can include machine based regression learning units/procedures and such approximate noise free projection data as the target (e.g., truth image) during training. Machine learning based regression models are well known. Embodiments of a CBCT imaging system including trained machine based regression learning units can be subsequently used to image subjects during normal imaging operations.

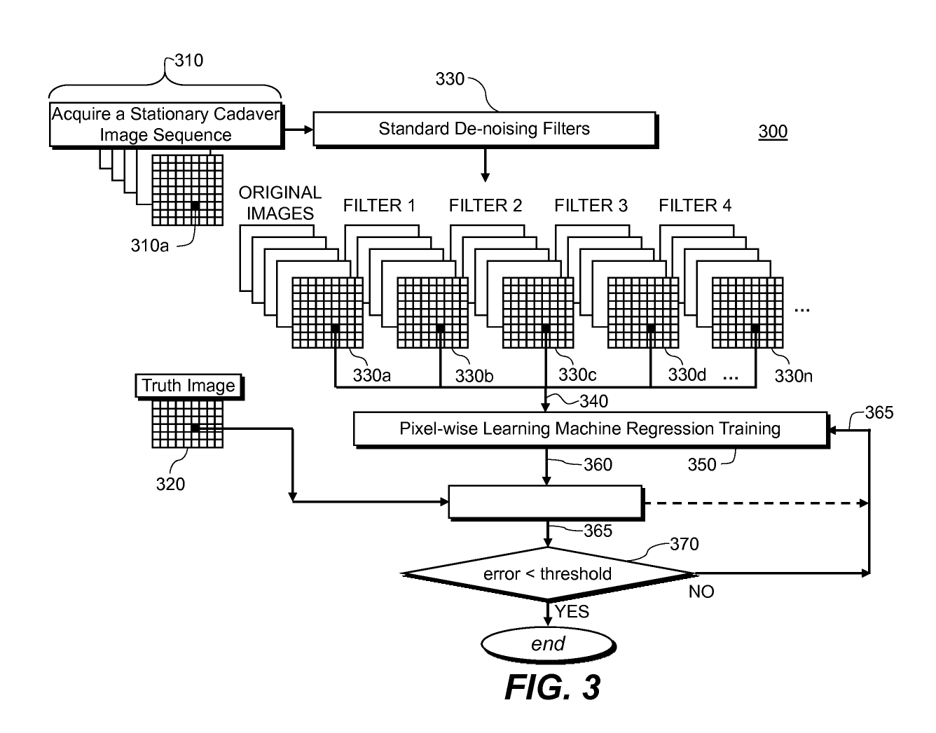

Architecture of an exemplary machine based regression learning unit that can be trained and/or used in embodiments of CBCT imaging systems according to the application is illustrated in FIG. 3. As shown in FIG. 3, an exemplary CBCT imaging system 300 can be used for the system 100 and can include a computational unit such as a machine based regression learning unit 350 and associated de-noising filters 330. As shown in FIG. 3, during training operations, the CBCT imaging system 300 can train the machine based regression learning unit 350 for later use with imaging operations of a CBCT imaging system. For example, the machine based regression learning unit 350 can be trained and later used by the same CBCT imaging system. Alternatively, the machine based regression learning unit 350 can be trained and later used by a CBCT imaging system using the same x-ray source (e.g., filtration such as Cu/Al and preferably under identical kVp). Alternatively, the machine based regression learning unit 350 can be trained and later used by the same type CBCT imaging system or same model CBCT imaging system. During such later imaging operations in the CBCT imaging system, the machine based regression learning unit 350 can decrease noise in transformed 2D projection data, in 3D reconstructed radiographic images and/or maintain image quality characteristics such as SNR, CNR or resolution (e.g., of resultant reconstructed volumes) at a reduced x-ray dose of the CBCT imaging system 300.

As shown in FIG. 3, a “truth image” 320 (e.g., low noise target image) can be obtained. As used herein, the “truth image” is an approximate noise free target image or noise reduced target data. For example, the truth image 320 can be obtained by comparing noise in a prescribed number (e.g., 1000) of projection images 310, in which an object is exposed preferably with identical x-ray parameter settings. For example, the object can be cadaver limb, cadaver knee, etc. imaged by the CBCT imaging system 300 for a complete scan (e.g., 200 degrees, 240 degrees, 360 degrees) of the object. Randomness of the noise in the plurality of images 310 used to form the truth image 320 can be statistically determined and then reduced or removed, for example, by averaging the 1000 images 310. Alternatively, instead of averaging, alternative analysis or statistical manipulation of the data such as weighting can be used to reduce or remove noise to obtain the truth image 320 or the approximate noise free target image (e.g., approximate noise free projection data).

In one embodiment, the truth image 320 and the projection images 310 can be normalized (e.g., from 0 to 1 or −1 to 1) to improve the efficiency of or simplify computational operations of the machine based regression learning unit 350.

After the truth image 320 is obtained, iterative training of the machine based regression learning unit 350 can begin. In one embodiment, one of the 1000 images 310 can be chosen and sent through a prescribed number such as 5 or more de-noising filters 330. Alternatively, embodiments according to the application can use 3, 7, 10 or 15 de-noising filters 330. For example, such exemplary de-noising filters 330 can be state-of-art de-noising filters that include but are not limited to Anisotropic Diffusion Filters, Wavelet Filters, Total Variation Minimization Filters, Gaussian Filters, or Median Filters. Outputs of the de-noising filters 330 as well as with the original image can be inputs 340 to the machine based regression learning unit 350 whose output can be compared to a target or the truth image 320. As shown in FIG. 3, the original image and outputs from filters (330b, 330c, 330d, . . . , 330n) are included in inputs 340. In one embodiment, an output 360 of the machine based regression learning unit 350 can be compared with the truth image 320 and an error 365 can be back-propagated into the machine based regression learning unit 350 to iteratively adjust node weighting coefficients, which connect inputs and output(s) of the machine based regression learning unit 350. For example, the machine based regression learning unit 350 can be implemented by a support-vector-machine (SVM) based regression learning unit, a neural network, interpolator or the like.

During exemplary training operations, the system 300 can process a projection image 310a one pixel at a time. In this example, the output of a SVM based regression learning machine as the machine based regression learning unit 350 can be a single result that is compared with the target and the error 365 can be back-propagated into the SVM based regression learning machine to iteratively adjust the node weighting coefficients connecting inputs and output of the SVM based regression learning machine to subsequently reduce or minimize the error 365. Alternatively, as each pixel in the input projection image 310a, 310b, 310n is processed by the machine based regression learning unit 350, a representation of the error 365 such as the error derivative can be back-propagated through the machine based regression learning unit 350 to iteratively improve and refine the machine based regression learning unit 350 approximation of the de-noising function (e.g., the mechanism to represent image data in the projection domain).

Completion of the machine based regression learning unit 350 training operations can be variously defined, for example, when the error 365 is measured 370. For example, the error 365 can be compared to a threshold including below a first threshold or a difference between subsequent iterations for the error 365 is below a second threshold, or a prescribed number of training iterations or projection images have been processed. Then, training operations for the machine based regression learning unit 350 can be terminated.

Training of the machine based regression learning unit 350 can be done on an object different than a subject being scanned during operational use of the machine based regression learning unit 350 in normal imaging operations of the CBCT imaging system 300. In one embodiment, the training can be done on a corresponding feature (e.g., knee, elbow, foot, hand, wrist, dental arch) of a cadaver at a selected kVp. Further, in another embodiment, the training can be done on a corresponding range of feature sizes or corresponding cadavers (e.g., male, adult, female, child, infant) at the selected kVp.

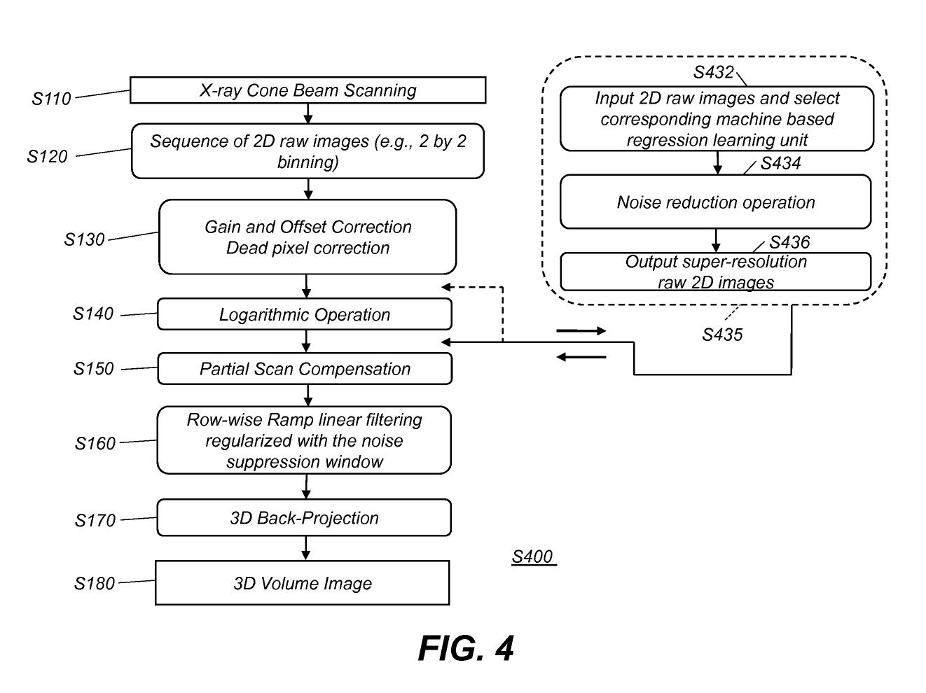

Referring to the logic flow diagram of FIG. 4, there is shown an image processing sequence 5400 according to an embodiment of the application. As shown in FIG. 4, the image processing sequence can be used for 3D volume image processing (e.g., CBCT). Steps S110, S120, S130, S140, S150, S160, in this sequence are the same steps described earlier for the conventional sequence of FIG. 2. In this exemplary sequence, a noise reduction process S435, indicated in dashed outline in FIG. 4, can follow image correction step S130 or follow the logarithmic operation step S140 and can input raw 2D image data and output transformed raw 2D image data comprising an increased SNR, and/or output transformed raw 2D image data including noise reduction or suppression.

As shown in FIG. 4, when a low dose mode or noise reduction mode is selected for a standard examination, a machine based regression learning unit for the corresponding examination (e.g., body part, exposure levels, etc.) can be selected in step S432. Then, the raw 2D image data from the detector can be passed through the selected machine based regression learning unit trained on the corresponding object to determine transformed raw 2D image data having a decreased noise in step S434. Then, the transformed raw 2D image data can be output for remaining volume image reconstruction processing in step S436.

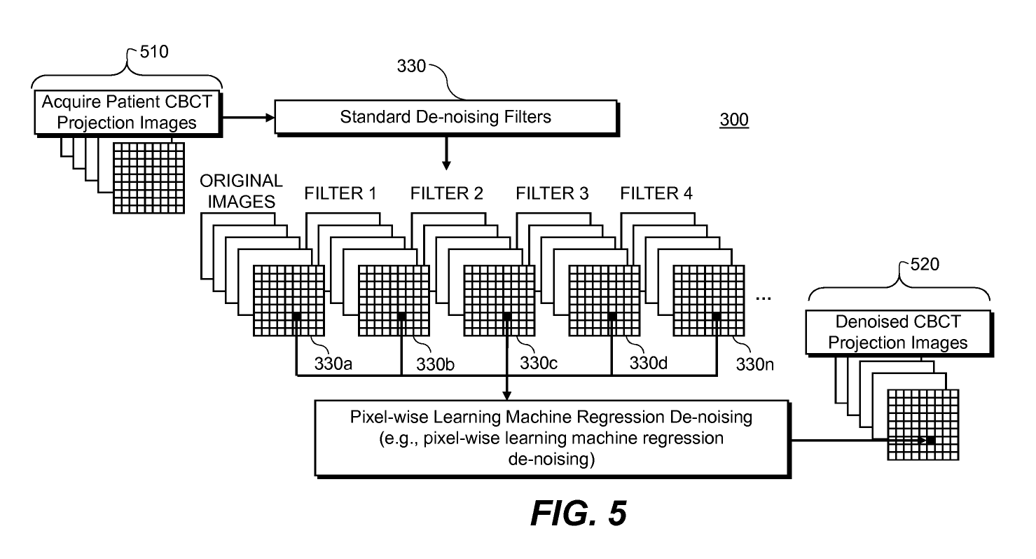

FIG. 5 is a diagram that shows an exemplary implementation of the process 425 using the machine based regression unit 350 in the CBCT imaging system 300. As shown in FIG. 5, raw 2D radiographic image data from a DR detector 510 can be passed though the plurality of de-noising filters 330 and outputs therefrom are input to the machine based regression unit 350. The machine based regression unit 330 can determine the appropriate pixel data from the original input data (e.g., 330a) and/or one of the de-noising filter outputs (e.g., 330b, . . . , 330n) for use as the reduced-noise or de-noised output data 520. Alternatively, the machine based regression unit 330 can select a combination of inputs or a weighted combination of inputs to be the output data 520 having the reduced noise characteristics. The raw 2D radiographic image data 510 from a DR detector is preferably corrected for gain and offset correction and the like before applying the de-noising according to embodiments of the application. Thus, the mechanism of a machine based regression learning unit can implement noise reducing imaging procedures for the CBCT imaging system 300 in the projection domain.

Machine learning based regression is a supervised parametric method and is known to one of ordinary skill in the art. Mathematically, there is an unknown function G(x) (the “truth”), which is a function of a vector \vec{x}. The vector \vec{x}^{t}=[x₁, x₂, . . . , x_{d}] has d components where d is termed the dimensionality of the input space. F(\vec{x}, \vec{w}) is a family of functions parameterized by \vec{w}. ŵ is the value of \vec{w} that can minimize a measure of error between G(x) and F(\vec{x}, \vec{w}). Machine learning is to estimate \vec{w} with ŵ by observing the N training instances vⱼ, j=1, . . . , N. The trained \vec{w} can be used to estimate the approximate noise-free projection data to achieve the purpose for low dose de-noising. According to embodiments of the application, because the attenuation coefficient is energy dependent, the estimated \vec{w} has to be energy dependent as well by conducting repeated measurements under different X-ray tube kVp and/or filtration settings. The trained \vec{w} is preferably a function of kVp, in that the selection of \vec{w} is preferably decided by the X-ray tube kVp in clinical application. Based on the first statement made above, a cadaver can be employed for training since the line integral variation from cadaver can be consistent with corresponding part in live human body.

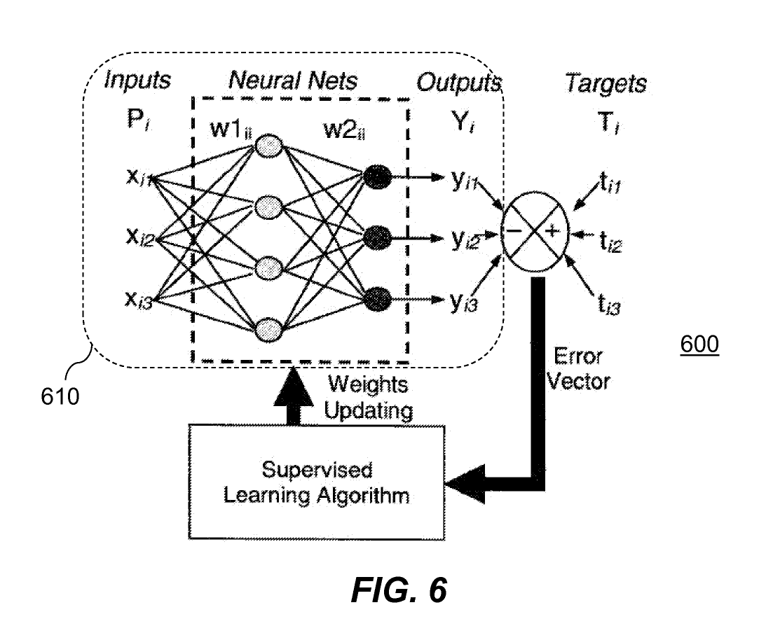

FIG. 6 is a diagram that shows a topological flow diagram of exemplary artificial neural networks that can be used in embodiments according to the application. Thus, an exemplary NN 610 shown in FIG. 6 can be used for the machine based regression learning unit 350, although embodiments are not intended to be limited thereby. An artificial neural network is a system based on the operation of biological neural networks, in other words, is an emulation of biological neural systems. A NN basically includes an input layer, hidden layers, an output layer and outputs as shown in FIG. 6.

A basic NN topological description follows. An input is presented to a neural network system 600 shown in FIG. 6 and a corresponding desired or target response is set at the output (when this is the case the training is called supervised). An error is composed from the difference between the desired (e.g., target) response and the NN output. Mathematically, the relationship between the inputs and outputs can be described as:

$${y}_{ij} = tanh ( {\sum }_{j = 1}^{4} w {2}_{ij} {Z}_{j} ) , \\ where {Z}_{i} = tanh ( {\sum }_{j = 1}^{3} w {1}_{ij} {X}_{ij} )$$

In the expression above, tanh is called an activation function that acts as a squashing function, such that the output of a neuron in a neural network is between certain values (e.g., usually between 0 and 1 or between −1 and 1). The bold black thick arrow indicates that the above NN system 600 is feed-forward back-propagated network. The error information is fed back in the NN system 600 during a training process and adaptively adjusts the NN 610 parameters (e.g., weights connecting the inputs to the hidden node and hidden nodes to the output nodes) in a systematic fashion (e.g., the learning rule). The process is repeated until the NN 610 or the NN system 600 performance is acceptable. After the training phase, the artificial neural network parameters are fixed and the NN 610 can be deployed to solve the problem at hand.

According to exemplary embodiments, the machine based regression learning unit 350 can be applied to projection images acquired through the DR detector using a CBCT imaging system and that application can result in decreased noise for the resulting image or a decreased x-ray dose (e.g., decreased mAs) can provide sufficient image resolution or SNR for diagnostic procedures. Thus, through the application of the trained machine based regression learning unit 350, an exemplary CBCT imaging system using a decreased x-ray dose can achieve a clinically acceptable image characteristics while other exposure parameters can be maintained.

According to exemplary embodiments, a trained noise reducing machine based regression learning unit as shown in FIG. 5 can be applied to any projection images acquired through a DR detector using a CBCT imaging system and that application can result in decreased noise in the resulting image or a lower-dose exposure achieving a prescribed SNR. Thus, through the application of the trained machine based regression learning unit, a current CBCT imaging system using a lower dose exposure setting can achieve the SNR resolution of a second higher exposure dose while other exposure parameters can be maintained. De-noised by the machine based regression learning unit according to the application can result in 2-D projection image data with improved characteristics.

Embodiments of the application can be used to generate a de-noised 2D projection data for each of a plurality of kVp settings and/or filtration settings (e.g., Al, Cu, specific thickness) for a corresponding examination. For example, when a wrist x-ray can be taken using 100 kVp, 110 kVp or a 120 kVp settings, a corresponding CBCT imaging system can use a machine based regression learning unit 350 trained for each of the three settings of kVp, however, a plurality of exposure settings can be trained using a single truth image. In one perspective, the machine based regression learning unit can be considered to have a selectable setting (e.g., corresponding training) for each of a plurality of exposure settings (e.g., kVp and/or filtration settings) for an examination type.

In one exemplary embodiment, a single individual view can be used to train the machine based regression learning unit 350 within a complete scan of the CBCT imaging system. In another exemplary embodiment, each of a plurality of individual views can be used to train the machine based regression learning unit 350 within a complete scan of the CBCT imaging system. For example, the machine based regression learning unit 350 can be trained using a truth image 320 for each 10 degrees of an exemplary CBCT imaging system scan. An exemplary CBCT imaging system scan can result in a prescribed number of raw 2D images, and alternatively the machine based regression learning unit 350 can be trained every preset number of the prescribed raw 2D images. Further, the CBCT imaging system can use a complete 360 degree scan of a subject or an interrupted 200-240 degree scan of the subject. In addition, the CBCT imaging system 300 can scan a weight bearing limb or extremity as the object.

Because of large variations and complexity, it is generally difficult to derive analytic solutions or simple equations to represent objects such as anatomy in medical images. Medical imaging tasks can use learning from examples for accurate representation of data and knowledge. By taking advantage of different strengths associated with each state-of-art de-noising filter as well as the machine learning technique, embodiments of medical imaging methods and/or systems according to the application can produce superior image quality even with low X-ray dose thus implement low dose X-ray cone beam CT imaging. Exemplary techniques and/or systems disclosed herein can also be used for X-ray radiographic imaging by incorporating the geometrical variable parameters into the training process. According to exemplary embodiments of system and/or methods according to the application, reduced noised projection data for exemplary CBCT imaging systems can produce corrected 2D projection image to include a SNR of an exposure dose 100%, 200% or greater than 400% higher.

Although described herein with respect to CBCT digital radiography systems, embodiments of the application are not intended to be so limited. For example, other DR imaging systems such as DR based tomographic imaging systems (e.g., tomosynthesis), dental DR imaging systems, mobile DR imaging systems or room-based DR imaging systems can utilize method and apparatus embodiments according to the application. As described herein, an exemplary flat panel DR detector/imager is capable of both single shot (radiographic) and continuous (fluoroscopic) image acquisition.

DR detectors can be classified into the “direct conversion type” one for directly converting the radiation to an electronic signal and the “indirect conversion type” one for converting the radiation to fluorescence to convert the fluorescence to an electronic signal. An indirect conversion type radiographic detector generally includes a scintillator for receiving the radiation to generate fluorescence with the strength in accordance with the amount of the radiation.

Cone beam CT for weight-bearing knee imaging as well as for other extremities is a promising imaging tool for diagnosis, preoperative planning and therapy assessment.

It should be noted that the present teachings are not intended to be limited in scope to the embodiments illustrated in the figures.

As used herein, controller/CPU for the detector panel (e.g., detector 24, FPD) or imaging system (controller 30 or detector controller) also includes an operating system (not shown) that is stored on the computer-accessible media RAM, ROM, and mass storage device, and is executed by processor. Examples of operating systems include Microsoft Windows®, Apple MacOS®, Linux®, UNIX®. Examples are not limited to any particular operating system, however, and the construction and use of such operating systems are well known within the art. Embodiments of controller/CPU for the detector (e.g., detector 12) or imaging system (controller 34 or 327) are not limited to any type of computer or computer-readable medium/computer-accessible medium (e.g., magnetic, electronic, optical). In varying embodiments, controller/CPU comprises a PC-compatible computer, a MacOS®-compatible computer, a Linux®-compatible computer, or a UNIX®-compatible computer. The construction and operation of such computers are well known within the art. The controller/CPU can be operated using at least one operating system to provide a graphical user interface (GUI) including a user-controllable pointer. The controller/CPU can have at least one web browser application program executing within at least one operating system, to permit users of the controller/CPU to access an intranet, extranet or Internet world-wide-web pages as addressed by Universal Resource Locator (URL) addresses. Examples of browser application programs include Microsoft Internet Explorer®.

In addition, while a particular feature of an embodiment has been disclosed with respect to only one or several implementations, such feature can be combined with one or more other features of the other implementations and/or combined with other exemplary embodiments as can be desired and advantageous for any given or particular function. Furthermore, to the extent that the terms “including,” “includes,” “having,” “has,” “with,” or variants thereof are used in either the detailed description and the claims, such terms are intended to be inclusive in a manner similar to the term “comprising.” The term “at least one of” is used to mean one or more of the listed items can be selected. Further, in the discussion and claims herein, the term “exemplary” indicates the description is used as an example, rather than implying that it is an ideal.

The invention has been described in detail with particular reference to exemplary embodiments, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A method for digital radiographic 3D volume image reconstruction of a subject, executed at least in part on a computer, comprising:
obtaining image data at a first examination setting for a plurality of first 2D projection images over a range of scan angles;
generating, for each of the plurality of first 2D projection images, a corresponding second 2D projection image by:
concurrently passing each of the plurality of 2D projection images through a plurality of de-noising filters;
providing a low noise image representation of a different corresponding object;
determining an image data transformation for the first examination setting according to the image representation using outputs of the plurality of de-noising filters and the low noise image representation of a different corresponding object;
applying the image data transformation individually to the plurality of first 2D projection images to generate the corresponding plurality of second 2D projection images; and
storing the plurality of second 2D projection images in a computer-accessible memory.

2. The method of claim 1 wherein the transformed plurality of second 2D projection images comprises a lower noise 2D projection images, higher SNR 2D projection images or higher CNR 2D projection images than the plurality of first 2D projection images.

3. The method of claim 1 wherein the image data transformation is provided by a computational unit, a neural network interpolator, a plurality of neural network interpolators, a machine-based regression learning device or a SVM machine regression learning device.

4. The method of claim 3 wherein the machine-based regression learning unit is based on an examination type or x-ray radiation source exposure setting.

5. The method of claim 3 wherein the image data transformation is angularly independent.

6. The method of claim 1 wherein the reduced noised projection data for the current 2D projection image comprises a SNR of an exposure dose 100%, 200% or greater than 400% higher.

7. The method of claim 1 wherein applying the image data transformation individually to the plurality of first 2D projection images comprises weighting a plurality of outputs of the plurality of de-noising filters,
wherein the machine-based regression learning unit is configured to operate on a pixel-by-pixel basis.

8. The method of claim 1 further comprising processing the transformed plurality of second 2D projection images to reconstruct the 3D volume image reconstruction of the subject.

9. The method of claim 1 wherein determining an image data transformation for the first examination setting comprises training a machine-based regression learning unit by:
determining a first image of a corresponding object;
passing scanned projection data of the corresponding object for a prescribed examination setting through the plurality of de-noising filters;
inputting the de-noised data from the plurality of de-noising filters into a machine-based regression learning unit to obtain a second estimated image of the corresponding object;
determining a difference between the second estimated image of the corresponding object and the first image; and
iteratively processing the de-noised data from the plurality of de-noising filters to determine an image data transformation to reduce the difference between the first image and the second estimated image.

10. The method of claim 9 wherein the training is completed for the prescribed examination setting when the difference for a projection image is less than a prescribed threshold, further comprising training for a plurality of prescribed examination settings.

11. The method of claim 9 wherein the training comprises training using a plurality of different corresponding objects.

12. The method of claim 1 wherein obtaining image data for the plurality of first 2D projection images comprises obtaining image data from a cone-beam computerized tomography apparatus or a tomography imaging apparatus.

13. The method of claim 1 further comprising:
processing the plurality of second 2D projection images to reconstruct a 3D volume image reconstruction of the subject;
displaying the 3D volume image reconstruction; and
storing the 3D volume image reconstruction in the computer-accessible memory, wherein the 3D volume image reconstruction is a orthopedic medical image, a dental medical image or a pediatric medical image.

14. The method of claim 13 wherein processing the processing the plurality of second 2D projection images comprises:
performing one or more of geometric correction, scatter correction, beam-hardening correction, and gain and offset correction on the plurality of 2D projection images;
performing a logarithmic operation on the plurality of 2D reduced noise projection images to obtain line integral data; and
performing a row-wise ramp linear filtering to the line integral data.

15. The method of claim 1 wherein the subject is a limb, an extremity, a weight bearing extremity or a portion of a dental arch.

16. The method of claim 1 wherein the image transformation is based on an examination type or x-ray radiation source exposure setting.

17. A method for digital radiographic 3D volume image reconstruction of a subject, executed at least in part on a computer, comprising:
obtaining cone-beam computed tomography image data at a prescribed exposure setting for a plurality of 2D projection images over a range of scan angles;
generating, for each of the plurality of 2D projection images, a lower noise projection image by:
(i) providing an image data transformation for the prescribed exposure setting according to image data from a different corresponding subject based on a set of noise-reducing filters;
(ii) applying the image data transformation individually to the plurality of 2D projection images obtained by:
(a) concurrently passing each of the plurality of 2D projection images through the set of noise-reducing filters; and
(b) applying the image data transformation individually to the plurality of first 2D projection images pixel-by-pixel to use the outputs of the set of noise-reducing filters to generate the corresponding plurality of lower noise projection images; and
storing the lower noise projection images in a computer-accessible memory.

18. A digital radiography CBCT imaging system for digital radiographic 3D volume image reconstruction of a subject, comprising:
a DR detector to obtain a plurality of CBCT 2D projection images over a range of scan angles at a first exposure setting;
a computational unit to generate, for each of the plurality of 2D projection images, an reduced-noise 2D projection image, the set of noise-reducing filters to select (i) an image data transformation for a prescribed exposure setting, a corresponding different subject, and a plurality of imaging filters, and (ii) apply the image data transformation individually to the plurality of 2D projection images obtained at the first exposure setting to generate the plurality of reduced-noise 2D projection images; and
a processor to store the reduced-noise plurality of 2D projection images in a computer-readable memory.

19. The digital radiography CBCT imaging system of claim 18, where the computational unit is a machine based regression learning unit.
